(19) United States
(12) Patent Application Publication
(10) Pub. No.: US 2026/0345459 A1
(43) Pub. Date: Nov. 5, 2026

(54) SUBMICRON PARTICLE COMPRISING A PEPTIDE ACTIVE, PREPARATION METHOD AND USES THEREOF, IN PARTICULAR COSMETIC USES

(71) Applicant: Sederma, Le Perray-en-Yvelines (FR)

(72) Inventors: Alex SAUNOIS, Le Perray-en-Yvelines (FR); Renata PLATCHECK RAFFIN, Le Perray-en-Yvelines (FR); Cristieli ROSSINI KOPP, Le Perray-en-Yvelines (FR)

(73) Assignee: Sederma, Le Perray-en-Yvelines (FR)

(21) Appl. No.: 19/488,316

(22) PCT Filed: Jun. 3, 2024
(86) PCT No.: PCT/EP2024/065152

§ 371 Date: Nov. 26, 2025

(30) Foreign Application Priority Data
Jun. 9, 2023 (FR) FR2305860

(51) Int. Cl. A61K 8/64; A61K 8/02; A61K 8/04; A61K 8/37; A61K 8/55; A61K 8/60; A61Q 19/00

(52) U.S. Cl. CPC A61K 8/64; A61K 8/025; A61K 8/044; A61K 8/37; A61K 8/553; A61K 8/602; A61Q 19/00; A61K 2800/10; A61K 2800/412; A61K 2800/591; A61K 2800/622; A61K 2800/652

(57) ABSTRACT

The submicron sized particle is substantially spherical, comprising an oily core containing the peptide active and a wax shell that is solid at room temperature. The particle is stabilized by an outer layer of a high-HLB non-ionic surfactant. The invention provides a method for obtaining an aqueous suspension of said particles with a satisfactory degree of encapsulation and improved long-term stability, for use in particular in the cosmetics industry. Peptide active associations can be considered, incorporated into the particles and adsorbed on the outside of the particles.

### CROSS REFERENCE TO RELATED APPLICATIONS

This application is the U.S. National Phase of PCT/EP2024/065152, filed Jun. 3, 2024, which claims priority to French Patent Application No. FR2305860, filed Jun. 9, 2023, the disclosures of these applications being incorporated herein by reference in their entireties for all purposes.

### REFERENCE TO AN ELECTRONIC SEQUENCE LISTING

The contents of the electronic sequence listing (PEPTCAPS-PCT.xml; Size: 16.4 KB; and Date of Creation: Oct. 17, 2025) is herein incorporated by reference in its entirety.

### TECHNICAL FIELD

The present invention relates to a submicron sized particle comprising a peptide active, being in particular a peptide or peptide mixture of the cosmetics field. It also relates to a method for preparing said particle, and to uses, particularly use for a non-therapeutic cosmetic treatment of the skin (including the scalp) and/or its integuments (such as body hair, eyelashes, eyebrows, nails or hair) of human or animal mammals, and use for manufacturing a topical or oral composition particularly intended for said treatment.

The invention mainly relates to the field of cosmetics and personal care and hygiene products that are applied topically.

### BACKGROUND OF THE INVENTION

Peptides, alone or as mixtures, are among the very popular biologically active ingredients in topical cosmetic formulations. Often consisting of fragments of amino acid sequences of proteins present in the skin, or derivatives of these fragments, they act in situ by biomimicry like these proteins.

Historic peptides, such as Matrixyl® or Matrixyl® 3000 (from Sederma), were developed for anti-ageing applications, particularly by stimulating the main proteins of the dermal extracellular matrix, collagen 1 and elastin. Today, many peptides are proposed, which can act on more varied targets and lead to cosmetic benefits highly awaited, such as moisturization, slimming, sensory skin comfort, radiance or pigmentation of skin, or else pigmentation of hair and bodily hairs.

A major advance in the field of cosmetic peptides consisted in derivatizing the peptide to modify its lipophilic nature and increase its bioavailability in the skin. Derivatization may be present in the C-terminal and/or N-terminal position of the peptide, formed, for example, by acylation, more particularly to attach a fatty chain to the amino acid sequence, particularly a C14 to C20 chain close to the chain lengths of skin lipids, preferentially a C16 palmitoyl (Pal) or C14 myristoyl (Myr) or C18 oleoyl carbon chain. The N-terminal Pal chain is often preferred, as it does not oxidize and has proven biological activity.

Other types of derivatizations have been proposed, such as binding a Biotinoyl moiety in place of the fatty chain, or binding derivatives of particular acids such as those of ascorbic, retinoic, cinnamic, oleanolic, hyaluronic, nicotinic, lipoic, gallic or pantothenic acid.

Amino acids derived in this manner are also used as active compounds in cosmetics, for example Oleyl-Tyrosine as a lightening compound, or Pal-Alanine, Pal-Glycine and Pal-Isoleucine as anti-ageing active agents (from the SEPPIC company).

The present invention relates more particularly to peptide actives of this type bearing a N-terminal and/or C-terminal derivatization, particularly derivatization of lipophilic nature, such as acylation with a palmitoyl chain.

Peptide actives may be sold in powder form (particularly acetate or hydrochloride salts) with a high degree of purity.

In the cosmetics industry, peptide actives are most often sold in the form of active ingredients, i.e. in the form of a pre-formula to be incorporated directly into the final cosmetic composition formulation.

This pre-formula corresponds to the peptide active agent dissolved in a solvent appropriate to the physicochemical characteristics of the peptide or mixture of peptides and/or amino acid or mixture of amino acids of the peptide active, such as glycerin, at a pH that allows the stability of the active in this pre-formula to be maintained.

One of the problems encountered is the long-term stability of the ingredient constituted by the pre-formula. It is not possible to store the ingredient at room temperature, but it is generally necessary to store it at 4/5 °C. According to the invention, “room temperature” means a temperature ranging from 15 to 30 °C.

### SUMMARY OF THE INVENTION

The aim of the present invention is to overcome this drawback, namely, to offer a solution for stabilizing amino acids or peptides, which are particularly lipophilic in chemical nature or by derivatization, so that they can be readily stored and handled at room temperature to realize cosmetic formulations, without any risk of degradation and loss of activity.

The present invention is also directed towards providing a means for improving the stability of the peptide active so that it retains optimum biological activity until it reaches its target in the skin or its integuments, while being protected from oxidizing and proteasic attacks.

Furthermore, the invention is directed towards providing a solution using environmentally friendly starting materials, particularly by banning ethoxylated surfactants.

To this end, according to a first aspect, the invention provides a submicron particle comprising a peptide active consisting of a peptide or peptide mixture and/or an amino acid or amino acid mixture, characterized in that said particle is substantially spherical, consisting of an oily core containing said peptide active surrounded by a shell of wax that is solid at room temperature, at least one non-ionic surfactant with an HLB substantially greater than or equal to 10 forming an outer layer on said wax shell.

According to a second aspect, the present invention provides a method for preparing particles according to the first aspect. To this end, a method is provided for encapsulating a peptide active consisting of a peptide or peptide mixture and/or an amino acid or amino acid mixture, comprising the following successive steps:
a step of providing a mixture comprising:
an aqueous phase in which the peptide active is dissolved, optionally by means of a water-miscible co-solvent and/or a pH adjuster,
a wax phase that is solid at room temperature, consisting essentially of one or more waxes; and
an oily phase that is liquid at room temperature, consisting essentially of one or more oils, the mixture also comprising at least one non-ionic surfactant (TA1) with an HLB substantially greater than or equal to 10, and each phase being rendered homogeneous and liquid where appropriate optionally by heating and/or by mechanical and/or physical means; and
a step of hot forming an emulsion of said mixture;
a step of reducing the particle size of said emulsion to a substantially submicron size; and
a cooling step in order to form a suspension of submicron-sized structured lipid particles in which said peptide active agent is encapsulated.

### BRIEF DESCRIPTION OF THE DRAWINGS

FIG. 1 schematically illustrates the morphology of the particles according to the invention.

FIG. 2 is a Differential Scanning calorimetry (DSC) analysis graph of the Pal-KTTKS encapsulated according to the invention.

FIG. 3 is a graph of the trypsin digestion kinetics of a Pal-KTTKS suspension encapsulated according to the invention.

FIG. 4 is an image obtained by transmission electron microscopy (TEM) of aPal-KTTKS suspension encapsulated according to the invention.

FIG. 5 is an image obtained by atomic force microscopy (AFM) of a Pal-KTTKS suspension encapsulated according to the invention.

FIG. 6 is an image obtained by transmission electron microscopy (TEM) of a Pal-GHK suspension encapsulated according to the invention.

FIG. 7 is a graph showing the rate of release of Pal-GHK encapsulated at 100 ppm as a function of time compared to that of free Pal-GHK.

FIG. 8 is a graph showing the rate of release of Pal-GHK encapsulated at 1000 ppm as a function of time compared to that of free Pal-GHK.

### DETAILED DESCRIPTION OF THE INVENTION

According to the invention, the term “peptide active” means a peptide or peptide mixture, said peptide comprising at least two amino acids, linked by an amide bond between one of their acid and amine function. The peptide may be of synthetic or natural origin, obtained by extraction or via a biotechnological route. It may be linear, cyclic, or branched. By extension, according to the invention, the term “peptide active” also comprises an amino acid or a mixture of free amino acids.

According to the invention, the term “amino acid” means a natural or synthetic amino acid, or one obtained by a biotechnological route, having L, D, or racemic stereochemistry. It may be chosen from natural amino acids, derivatives thereof (for example hydroxyproline) or analogues (for example 5-hydroxyproline or methionine sulfone or sulfoxide). The acid and amine functions may be separated by one or more carbons, one carbon corresponding to natural alpha-amino acids, two carbons corresponding to beta-amino acids (for example beta-alanine), three carbons to gamma-amino acids (for example GABA or gamma-aminobutyric acid), four carbons corresponding to delta-amino acids (for example 5-aminovaleric acid), etc.

The submicron particles according to the invention comprise two types of lipids: lipids that are solid at room temperature and lipids that are liquid at room temperature. The particles are of SLC type meaning “solid liquid nanocarrier”. According to the invention, mixing of the two types of lipids results in particles that are solid at room temperature, thanks to a substantially waxy, and therefore solid, shell, and an oily core in which the peptide active agent is trapped, protected from the outside by the waxy shell. The term “structured” is used to characterize particles that are solid at room temperature (visible by Differential Scanning calorimetry (DSC) analysis, see the below detailed description showing for example a melting temperature well above 30 °C for particles encapsulating the Pal-KTTKS peptide).

The term “submicron particles” is used according to the invention for particles with a submicron mean diameter, i.e. less than 1 μm (=1000 nm), and not the term “nanoparticles”, as the particles according to the invention do not correspond to the WTO definition and European Union regulations, which limit the term “nano-material” to insoluble particles with a nominal diameter ranging from 1 to 100 nm. The term “mean” submicron diameter corresponds to the mean particle diameter at the 50th percentile of the distribution curve (D50).

Test results given below in the detailed description show the structured lipid particles formed according to the method of the invention with a set of representative cosmetic peptides, the encapsulation efficiency and the particle stability over time at room temperature.

The results particularly show:
that structured submicron sized particles are obtained, which are spherical and homogeneous at room temperature, without aggregation and phase separation during manufacture, following a Gaussian size profile around a mean value of 100 to 900 nm, with low polydispersity (less than or equal to 2), a high degree of encapsulation, and that can comprise a content after encapsulation that is compatible with use in formulating cosmetic products (up to 5000 ppm);
that the particles are stable, without aggregation and phase separation, without releasing peptide compound(s) at room temperature for several months, and up to 1 year for the Pal-KTTKS;
that the particles are also stable, without aggregation and phase separation, without releasing peptide compound(s) at 40 °C, stimulating accelerated stability;
a slow-release profile of the peptide compound(s) is obtained, with a half-life suitable for topical delivery, i.e. the stability of the peptide active is maintained during the period of release into the skin;
from a formulation point of view, as the peptide active is encapsulated and protected in a particle with a waxy shell, it will be less sensitive to electrolytes, and the risk of undesirable precipitation in the presence of certain adjuvants, such as carbomers, will be limited or even eliminated;
that environmentally friendly starting materials can be used, particularly non-ethoxylated surfactants such as lauryl glucoside or decyl glucoside.

A further advantage according to the invention lies in the fact that the penetration of peptide compounds through the stratum corneum will be enhanced, down to deeper layers of the skin, as the lipid composition of the particles and their very small size (submicron) contribute towards increasing the adhesion of the particles and their contact surface with the stratum corneum thanks to a lipid rearrangement.

Yet another advantage of the method according to the invention is that industrialization is simple. Organic solvents can be avoided, and production times are short. High-pressure homogenizers are commonly used in many industries, particularly in the cosmetics industry, to obtain submicron particles.

According to the invention, the wax is chosen to be solid at room temperature, i.e. having a melting point, alone or as a mixture of waxes, above 30 °C, preferably from 40 °C to 80 °C, more preferably from 42 °C to 65 °C.

Preferably, the wax used is natural or synthetic, consisting of at least one ester of C12-C36 fatty acid(s) and of C12-C36 fatty alcohol(s), preferably chosen from triglycerides, diglycerides, monoglycerides, and from monoesters of C18-C36 fatty acid(s) and of C18-C36 fatty alcohol(s), more preferably cetyl palmitate, glyceryl tribehenate, glyceryl stearate or tristearate, a wax of plant origin, such as rice wax, or a wax of animal origin, such as beeswax. According to the invention, the wax is a solid lipid at room temperature.

According to the method, this wax phase is heated to the melting temperature of the wax or wax mixture and kept liquid at about this temperature for the steps of suspension formation and suspension particle size reduction.

Preferably, according to the invention, use is made of an oil or a mixture of oils, which by definition are liquid at room temperature, comprising:
essentially C8 to C18 fatty acids,
mono-, di- or tri-esters of essentially C8 to C18 fatty acids, comprising mono-, di- or tri-glycerides of fatty acids; and/or
fatty alcohols comprising C8 to C18 carbon chains.

Preferably, the oils are chosen from C8 to C18 fatty acid esters.

Preferably, the oils used according to the invention are of plant origin, particularly derived from soybean, rapeseed, sunflower, palm and/or coconut.

Preferably, in the method of the invention, the oily phase is heated to the same temperature as the waxy phase. Preferably also, the waxy phase and the oily phase are heated simultaneously.

According to the method of the invention, the peptide active may be added pure in the form of a powder directly to the aqueous phase, or pre-dissolved in a fraction of the aqueous phase. If this peptide active agent is insoluble or is only partially soluble in the aqueous phase, one or more water-miscible co-solvents in which the peptide compound or mixture of peptide compounds is soluble, such as glycerol or sorbitol, may be used.

According to the method of the invention, it may also be necessary, with or without co-solvent(s), to aid the dissolution of the peptide compound by adding a fraction of the oil or oil mixture, by applying mechanical means, such as stirring, or other means (microwave, ultrasound, electromagnetic and/or electrical waves), and/or by heating. It may also be necessary to add a pH adjuster, such as citric acid or NaOH, to the aqueous phase to acidify or alkalize the medium and aid dissolution.

According to the invention, it may also be advantageous to use a second surfactant (TA2) with a low HLB, substantially less than or equal to 6. This second surfactant will promote particle stabilization (more stable size over time and at temperatures above 40 °C). According to preferred characteristics, this second surfactant is used. The particles according to the invention preferably comprise this second surfactant.

According to other preferred and advantageous features of the particles and/or the method of the invention: (the weight percentages being expressed relative to the total weight of the mixture or suspension):
the weight percentage of the wax or wax mixture is comprised between 1 and 15, preferably between 1 and 10, more preferably between 2 and 8; and/or
the weight percentage of the oil or oil mixture is comprised between 2 and 20, preferably between 5 and 15, more preferably between 7.5 and 12.5; and/or
the ratio of the weight percentage of wax to the weight percentage of oil in the particles is comprised between 1:1 and 1:10, preferably between 1:2 and 1:8, more preferably between 1:2 and 1:5; and/or
the weight percentage of water is comprised between 30 and 90, preferably between 45 and 90, more preferably between 50 and 85; and/or
the first surfactant TA1 preferably has an HLB of greater than or equal to 12; and/or
the second surfactant TA2, when present, preferably has an HLB of between 3.5 and 5.5; and/or
the first surfactant TA1 preferably comprises at least one alkylglucoside or alkylpolyglucoside of at least one saturated C10-C16 fatty alcohol, and/or at least one polyglycerol ester of a saturated C10-C16 fatty acid, and/or at least one polysorbate (esters of ethoxylated sorbitans), a poloxamer (formed from chains of ethylene oxide and propylene oxide blocks), and/or a polyvinyl derivative (consisting of vinyl acetate and vinylpyrolidone, such as polyvinyl alcohol PVA or polyvinylpyrrolidone PVP); and/or
preferably the at least one alkyl glucoside or alkyl polyglucoside of at least one saturated C10-C16 fatty alcohol is chosen from lauryl glucoside, decyl glucoside, coco glucoside and polyglucoside derivatives thereof, more preferably chosen from lauryl glucoside and decyl glucoside, and/or
preferably the at least one polyglycerol ester of a saturated C10-C16 fatty acid is chosen from polyglyceryl-4 to -6 lauric, sebacic, caprylic or capric acid, or mixtures thereof, preferably the mixture polyglyceryl-4 laurate/sebacate and polyglyceryl-6 caprylate/caprate, or the mixture polyglyceryl-4 laurate/sebacate and polyglyceryl-4 caprylate/caprate; and/or
preferably, the polysorbate is chosen from polysorbate 60 and polysorbate 80; and/or
preferably, the poloxamer is poloxamer 188; and/or
preferably, the polyvinyl derivative is polyvinyl alcohol; and/or
the first surfactant TA1 is added to the aqueous phase, where appropriate optionally dissolved by heating; and/or
the second surfactant TA2 is chosen from a phospholipid or a monoester, particularly of sorbitan, or a mixture of the two, the phospholipid preferably being chosen from phosphatidic acid, a phosphatidylcholine or lysophosphatidylcholine, a glycerophosphocholine, phosphatidylserine or lysophosphatidylserine, phosphatidylethanolamine, phosphatidylinositol or sphingomyelin, the second surfactant more preferably being hydrogenated, and more preferentially is hydrogenated lecithin, the second surfactant TA2 being a phospholipid, it is preferably added to the waxy phase, where appropriate optionally dissolved by heating, the second surfactant TA2 being a sorbitan monoester, it is preferably chosen from sorbitan monostearate, sorbitan sesquistearate, sorbitan laurate, sorbitan oleate, sorbitan sesquioleate and sorbitan isostearate, more preferably sorbitan isostearate, preferably added to the wax phase, dissolved by heating; and/or
a natural or synthetic wax is used, preferably consisting of at least one ester of C12-C36 fatty acid(s) and of C12-C36 fatty alcohol(s), more preferably chosen from triglycerides, diglycerides, monoglycerides, and monoesters of C18-C36 fatty acid(s) and of C18-C36 fatty alcohol(s), more preferentially cetyl palmitate, glyceryl tribehenate, glyceryl stearate or tristearate, a wax of plant origin, such as rice wax, or a wax of animal origin, such as beeswax; and/or
an oil is used which is preferably formed from esters of essentially C16-C18 fatty acid(s), particularly monoesters such as ethyl oleate, or triglycerides such as the caprylic/capric triglyceride mixture; and/or
the weight percentage of the first surfactant TA1 is from 1% to 6%, more preferably from 2% to 4%; and/or
the weight percentage of the second surfactant TA2 is from 0.1% to 5%, more preferably from 0.5% to 2%; and/or
the particle size reduction step is performed by high-pressure homogenization, membrane emulsification, micro-fluidization, using a rotor-stator of the Ultraturrax type; and/or
the method comprises the addition of one or more preserving agents and/or one or more antioxidants, and/or bacteriostatic agents, preferably from 0.1% to 5%, in one of the phases or as an additional phase, preferably in the aqueous phase, such as 1,2-octanediol, propanol, citric acid, hexanediol, propanediol, pentiol, sodium benzoate; and/or
the final pH of said suspension is preferably comprised between 3 and 9, which is compatible with a formulation for the skin and/or its integuments; it varies as a function of the peptide active; and/or
when a co-solvent is present, its amount is a maximum of 30%, said co-solvent being found mixed with the aqueous phase at the end of the method.

Optionally, the method comprises an additional drying step in which water is removed in order to obtain a dried suspension, which step may be performed by drying the aqueous phase to obtain a powder, for example by spray-drying or freeze-drying, particularly using a sugar such as mannitol or maltodextrin as a drying aid.

Preferably, the particles according to the invention are in aqueous suspension, which may where appropriate optionally contain a co-solvent used to help dissolve the peptide.

A particularly advantageous combination of the method according to the invention is characterized in that the mixture comprises or consists of:
1 to 5000 ppm of peptide active;
2% to 8% by weight of a wax or a mixture of waxes;
7.5% to 12.5% by weight of an oil or a mixture of oils;
2% to 4% by weight of lauryl glucoside or decyl glucoside;
optionally from 0.5% to 2% by weight of hydrogenated phosphatidylserine or phosphatidylcholine; and
optionally a co-solvent, a pH adjuster and/or a preserving agent and/or a bacteriostatic and/or antioxidant agent;
   all the weight percentages being expressed as a function of the total weight of the mixture or suspension, and the balance qs to 100% by weight being provided by the weight of water.

Optionally, the method according to the invention comprises a step of adding an additional peptide active to the suspension obtained after cooling and production of solid particles. The additional peptide active agent is pre-dissolved in water with addition of a water-miscible co-solvent if necessary, and is then added to and mixed with the suspension. This step advantageously allows a second peptide active to be adsorbed onto the outside of the particles and jointly transported to an active site.

Preferably, the free amino acid or free amino acids and/or peptide or peptides constituting the peptide active comprise a derivatization, preferably in the N-terminal or C-terminal position, the peptide active corresponding to the following general formula (A): X-(Xaa)n-Z in which:
Xaa is an amino acid, derivative or analogue;
n is an integer ≥1, when n≥2 the amino acids being chosen independently of each other;
N-terminal X chosen from H, —CO—R¹, —SO₂—R¹ or a biotinoyl group;
C-terminal Z chosen from OH, OR¹, NH₂, NHR¹ or NR¹R²; and
R¹ and R² being, independently of each other, chosen from an alkyl, aryl, aralkyl, alkylaryl, alkoxy, saccharide and aryloxy group, which may be linear, branched, cyclic, polycyclic, unsaturated, hydroxylated, carbonylated, phosphorylated and/or sulfurized, said group having from 1 to 24 carbon atoms and possibly having in its backbone one or more heteroatoms O, S and/or N;
the case where X═H and Z═OH being excluded.

Preferably, according to the invention, the peptide active comprises one or more peptides. More preferably, the peptide(s) of the peptide active contain a sequence of not more than 10 amino acids, corresponding in formula (A) to 2≤n≤10, more preferably a sequence of not more than 6 amino acids, corresponding in formula (A) to 2≤n≤6.

According to the invention, the peptide active, when it consists of a mixture of peptides, preferably comprises a mixture of 2 to 5 peptides, and more preferably a mixture of 2 peptides.

According to other preferred features of the invention, in formula (A):
R¹ and/or R² is an alkyl chain of 1 to 24 carbon atoms, preferably a lipophilic alkyl chain of 3 to 24 carbon atoms, more preferably 8 to 24 carbon atoms, and more preferentially 12 to 16 carbon atoms; and/or
X is an acyl group CO—R¹; preferably chosen from octanoyl (C8), decanoyl (C10), lauroyl (C12), myristoyl (C14), palmitoyl (C16), stearoyl (C18), biotinoyl, elaidoyl, oleoyl and lipoyl; more preferably chosen from lauroyl (C12), myristoyl (C14) and palmitoyl (C16), and/or
Z is chosen from OH, OMe, OEt and NH₂, preferably OH; and/or
X is chosen from palmitoyl (C16), myristoyl (C14), lauroyl (C12) or biotinoyl; more preferably palmitoyl (C16), and Z is OH.

Peptides comprising in the N- or C-terminal positions derivatives of particular acids such as ascorbic, retinoic, cinnamic, oleanolic, hyaluronic, nicotinic, lipoic, gallic or pantothenic acid are also covered by the present invention.

Preferably, the amino acids of the peptide active and/or of the peptide(s) forming it are thus chosen from the amino acids (L and/or D): histidine (H, His), arginine (R, Arg), lysine (K, Lys), phenylalanine (F, Phe), alanine (A, Ala), leucine (L, Leu), methionine (M, Met), isoleucine (I, Ile), tryptophan (W, Trp), proline (P, Pro), valine (V, Val), cysteine (C, Cys), glycine (G, Gly), glutamine (Q, Gln), asparagine (N, Asn), serine (S, Ser), tyrosine (Tyr, Y), threonine (T, Thr), aspartic acid (Asp, D) and glutamic acid (Glu, E), oxygenated derivatives thereof such as methionine sulfone or sulfoxide, or a hydroxyproline, and structural analogues thereof such as the lysine analogue ornithine, for example.

The peptide active according to the invention may be optically pure or consist of the L or D isomers thereof or a mixture thereof. The L isomers thereof, which are naturally occurring, may be preferred. It may be obtained via a synthetic or biotechnological route.

The peptide may be complexed with a metal ion (for example copper, zinc, manganese or magnesium).

The detailed description given below illustrates in detail all these advantages and also others obtained with the method of the invention. The following di-, tri-, tetra-, penta-, hexa-peptides representatives of the cosmetics are given by way of example.
The Pal-PP dipeptide (Palmitoyl Dipeptide-52) corresponding to the palmitoylated proline-proline peptide sequence. This peptide has pro-pigmenting activity and is particularly adapted to the prevention or treatment of canities. For example, this peptide is sold by the Applicant in solution form under the trade name Sylverfree™.
The Pal-GHK tripeptide (INCI name: Palmitoyl Tripeptide-1) corresponding to the palmitoylated glycine-histidine-lysine peptide sequence. This peptide stimulates fibroblast synthesis of important dermal extracellular matrix molecules, such as collagen and elastin. It allows the skin's mechanical properties—elasticity and firmness—to be reinforced, thereby helping to treat wrinkles. For example, this peptide is sold in solution form by the Applicant under the trade name Biopeptide CL™.
The Biot-GHK (INCI name: Biotinoyl Tripeptide-1) is the biotinoylated version of Pal-GHK. It also allows the synthesis of collagen IV and laminin 5 to be stimulated, and hair follicle activity to be stimulated. It is particularly recommended for preventing or treating hair loss. For example, this peptide is sold in solution form by the Applicant under the trade name Procapil™.
The Pal-KMO₂K (INCI name: Palmitoyl tripeptide-38) corresponds to the palmitoylated lysine-methionine-lysine peptide sequence, MO₂ corresponding to a dioxygenated methionine. This peptide stimulates the production of collagens I and II, fibronectin and hyaluronic acid by dermal fibroblasts. It is recommended for anti-ageing treatments, for example sold in solution form by the Applicant under the trade name Matrixyl® Synthe'6.
The Pal-GQPR (INCI name: Palmitoyl Tetrapeptide) (SEQ ID NO. 1) corresponds to the palmitoylated glycine-glutamine-proline-arginine peptide sequence. This peptide acts on excess interleukin production. It is adapted to inhibit damages, in particular damages linked to glycation. For example, it is sold by the Applicant in solution form under the trade name Rigin™. It is also sold in solution as a mixture with Pal-GHK by the Applicant under the trade name Matrixyl® 3000 for proven anti-synergetic ageing activity. Encapsulation of the Pal-GHK/Pal-GQPR mixture is also given by way of example later in the description.
The Pal-KTFK (INCI name: Palmitoyl Tetrapeptide-10) (SEQ ID NO. 2) corresponds to the palmitoylated lysine-threonine-phenyalanine-lysine peptide sequence. This peptide has epidermal activity, preserving the skin barrier, smoothing out the relief and particularly stimulating the chaperone protein α-crystallin for an effect on the transparency and radiance of the complexion. It is also described as stimulating the main dermal proteins. It is sold in solution form by the Applicant under the trade name Crystalide®.
The Pal-KTTKS (INCI name: Palmitoyl pentapeptide-4) (SEQ ID NO. 3) corresponds to the palmitoylated lysine-threonine-threonine-lysine-serine (KTTKS) peptide sequence. This peptide presents a very complete activity on dermis and epidermis. It is recommended as a global anti-ageing agent, particularly in solution form under the trade name Matrixyl® by the Applicant.
The Pal-VGVAPG (INCI name: Palmitoyl Hexapeptide-12) (SEQ ID NO. 4) corresponds to the palmitoylated valine-glycine-valine-alanine-proline-glycine peptide sequence. This peptide stimulates fibroblast mobility and acts on skin suppleness. It is recommended for improving skin firmness and tone. It is particularly sold by the Applicant in solution in the commercial product Dermaxyl®.

The invention may apply to peptides other than those mentioned above, particularly the Pal-KTSKS (SEQ ID NO. 5), the Pal-K(P)HG(K(P) meaning a proline grafted onto a lysine), a cyclic peptide derived from linseed as described in WO 2019/149150, the Acetyl-Tyr-Arg-hexadecyl ester (INCI name: Acetyl Dipeptide-1 Cetyl Ester, corresponding to the tyrosine-arginine peptide chain acetylated on the N-terminal end and esterified with a hexadecyl chain on the O-terminal end), the Myr-PPL, the Pal-LLAN (SEQ ID NO. 6), the Pal-YGGFL (SEQ ID NO. 7), the VW, the Pal-K-Ava-K, the Pal-Y(Me)-OH, the Pal-KHG or the Pal-KGH.

Numerous examples of cosmetic peptides are described in the Applicant's patent applications and patents to which the present invention may be applied. Amino acids or mixtures of amino acids may also be encapsulated according to the invention, particularly those mentioned above.

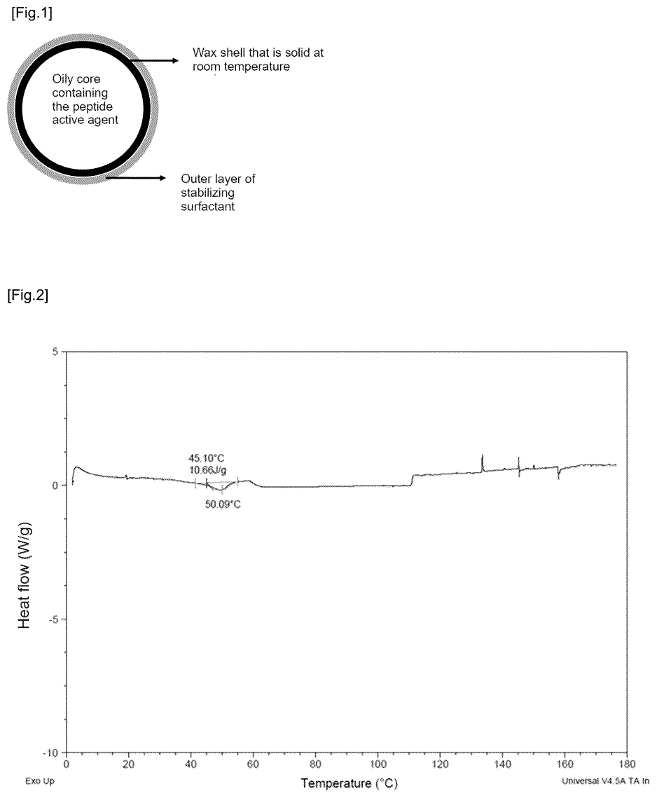

FIG. 1 schematically illustrates the predictive structure of the particles obtained: a wax shell surrounded by the first surfactant which stabilizes it, an oily core in which the peptide active is trapped, the second surfactant, when present, being located in the core of the particle and at the interface with the wax.

According to a third aspect, the present invention also provides an aqueous suspension of submicron-sized structured lipid particles according to the first aspect defined above, which may be obtained according to the method of the invention, as defined according to the second aspect defined above.

According to preferred and advantageous features of the suspension according to the invention:
the weight percentage of encapsulated peptide compound or mixture of peptide compounds relative to the weight percentage of said suspension is from 0.001% to 0.5%, preferably from 0.05% to 0.2%; more preferably from 0.01% to 0.1%; and/or
the overall particle size is from 0.1 to 1 μm, preferably from 0.15 to 0.6 μm, more preferably from 0.2 to 0.5 μm; and/or
the polydispersity index is from 0.1 to 2.0, preferably from 0.1 to 1.5, with a major mean particle size (D50) of between 0.2 and 0.9 μm, preferably between 0.2 and 0.4; and/or
the particles are substantially spherically structured and include a waxy shell; they also include an oily core containing said peptide active; and/or
the suspension is in a dried form (a powder), this form being advantageously suitable for formulating makeup products, for example.

An aqueous suspension according to the invention is water-dispersible and may be used to form cosmetic formulations of the emulsion, gel, serum or suspension type, for finished products for the skin and its integuments, such as haircare products (shampoo, conditioner).

According to a fourth aspect, the present invention provides a composition, particularly a cosmetic composition, for the skin and/or its integuments such as hair, but which may also be a pharmaceutical composition, comprising as active ingredient an effective amount of particles as defined according to the first aspect, or of a suspension, as defined above according to the third aspect, and a physiologically acceptable vehicle. Such a composition is preferably topical.

The term “physiologically acceptable” means that the compositions are suitable for topical or transdermal use, in contact with mucous membranes, nails, scalp, hair, bodily hairs and skin of mammals and more particularly humans, compositions that can be ingested or injected into the skin, without risk of toxicity, incompatibility, instability, allergic response, and the like. This “physiologically acceptable medium” forms what is conventionally known as the composition's excipient.

According to the invention, the term “topical treatment” or “topical use” refers to an application that is intended to act at the site where it is applied: skin, mucous membranes and/or integuments.

A composition according to the invention may be applied to the face, body or neckline in any form or vehicle known to those skilled in the art, particularly in the form of a solution, dispersion, emulsion, paste or powder.

In cosmetics in particular, applications may be proposed, particularly in facial and/or body skincare ranges and makeup-care ranges, particularly for the eyelashes or the eyebrows.

The composition may also be incorporated on a non-woven or woven material, made of natural or synthetic fibres, wool, or on any material intended to come into contact with the skin and which may be used in clothing, particularly tights and socks, shorties, day or night underwear, handkerchiefs, or fabrics, so as to exert its cosmetic effect via this skin/textile contact and allow continuous topical delivery (cosmeto-textiles).

The formulations may be included in personal care and/or beauty product ranges, particularly skincare, cleansing, makeup, makeup removal, anti-sun, artificial tanning, pre-shaving, shaving or aftershave ranges, moisturizers, humectants, emollients, conditioners, exfoliants, astringents, depilatories, antiperspirants, deodorants, etc.

According to preferential and advantageous features, the composition according to the invention may also comprise one or more other active ingredients.

The International Cosmetic Ingredient Dictionary & Handbook published by the Cosmetic, Toiletry, and Fragrance Association, Inc. (CTFA), Washington, D.C., describes a wide variety, without limitation, of cosmetic and pharmaceutical ingredients commonly used in the skincare industry, which are suitable for use as additional ingredients in compositions according to the present invention, as long as they are physically and chemically compatible with the other ingredients of the composition and especially with the active agents of the present invention. Moreover, the nature of these additional ingredients must not impair the benefits of the active agents of the invention. These additional ingredients may be synthetic or natural, such as plant extracts, or be derived from a biofermentation method.

Other skincare active agents that are particularly useful in combination with the composition according to the invention can be found in the sales literature of Sederma, Crodarom and Alban Muller and on the website www.croda.fr.

Mention may also be made, by way of example, of the following commercial active agents: betaine, glycerin, Actimoist Bio 2™ (Active Organics), AquaCacteen™ (Mibelle AG Cosmetics), Aquaphyline™ (Silab), AquaregulK™ (Solabia), Carciline™ (Greentech), Codiavelane™ (Biotech Marine), Dermaflux™ (Arch Chemicals, Inc), Hydra′Flow™ (Sochibo), Hydromoist L™ (Symrise), RenovHyal™ (Soliance), Seamoss™ (Biotech Marine), Argireline™ (trade name of acetyl hexapeptide-3 from Lipotec), spilanthol or an extract of Acmella oleracea known as Gatuline Expression™ (Gattefossé), an extract of Boswellia serrata known as Boswellin™, Deepaline PVB™ (SEPPIC), Syn-AKE™ (Pentapharm), Ameliox™, Bioxilift™ (Silab), PhytoCellTec™Argan (Mibelle), Papilactyl D™ (Silab), Preventhelia™ (Lipotec), or one or more of the following active ingredients sold by Sederma: Subliskin™, Venuceane™, Moist 24™, Vegesome Moist 24™, Essenskin™, Juvinity™, Revidrat™, Resistem™, Chronodyn™, Kombuchka™, Chromocare™, Calmosensine™, Glycokin factor ST, Biobustyl™, Idealift™, Ceramide 2™, Ceramide A2™, Ceramide HO3™, Legance™, Intenslim™, Prodizia™, Beautifeye™, Pacifeel™, Zingerslim™, Meiritage™, Sebuless™, Apiscalp™, Rubistem™, Citystem™, Neonyca™, NG Shea Butter Unsaponifiables™, Majestem™, Hydronesis™, Poretect™, Crystalide™, Amberstem™, Synchrolife™, Sylverfree™, Feminage™, Ameyezing™, Mel[o]stem™, or mixtures thereof.

Among the plant extracts (in the form of conventional extracts or prepared by an in vitro method) that may be used as additional active agents, mention may also be made, in particular, of extracts of ivy, for example climbing ivy (Hedera helix), Bupleurum chinensis, Bupleurum falcatum, arnica (Arnica montana L.), rosemary (Rosmarinus officinalis N.), calendula (Calendula officinalis), sage (Salvia officinalis L.), ginseng (Panax ginseng), Ginkgo biloba, St. John's wort (Hyperycum perforatum), butcher's broom (Ruscus aculeatus L.), meadowsweet (Filipendula ulmaria L.), orthosiphon (Orthosiphon stamincus Benth.), artichoke (Cynara scolymus), seaweed (Fucus vesiculosus), birch (Betula alba), green tea, kola nut (Cola nitida), horse chestnut, bamboo, Centella asiatica, heather, wrack, willow, pilosella, escin extracts, cangzhu extracts, Chrysanthellum indicum extracts, plants of the Armeniacea genus, Atractylodis platicodon, Sinnomenum, pharbitidis, Flemingia, coleus such as C. forskohlii, C. blumei, C. esquirolii, C. scutellaroides, C. xanthantus and C. barbatus, such as Coleus barbatus root extract, extracts of horehound, Guioa, Davallia, Terminalia, Barringtonia, Trema, Antirobia, Cecropia, Argania, Dioscoreae such as Dioscorea opposita or Dioscorea mexicana, extracts of Ammi visnaga, Siegesbeckia, in particular Siegesbeckia orientalis, plant extracts from the Ericaceae family, in particular extracts of bilberry (Vaccinium angustifolium), Arctostaphylos uva ursi, Aloe vera, sterol-containing plants (particularly phytosterols), Manjistha (extract of plants of the Rubia genus, in particular Rubia cordifolia), Guggal (extract of plants of the genus Commiphora, in particular Commiphora mukul), an extract of kola, chamomile, red clover, Piper methysticum (Kava Kava from Sederma), Bacopa monieri (Bacocalmine™, Sederma) and sea whip, Glycyrrhiza glabra, mulberry, Melaleuca (tea tree), Larrea divaricata, Rabdosia rubescens, Euglena gracilis, Fibraurea recisa hirudinea, Chaparral sorghum, sunflower, Enantia chlorantha, Mitracarpe of the Spermacocea genus, Buchu barosma, Lawsonia inermis L., Adiantium capillus-veneris L., Chelidonium majus, Luffa cylindrica, Japanese mandarin (Citrus reticulata blanco var. unshiu), Camelia sinensis, Imperata cylindrica, Glaucium flavum, Cupressus sempervirens, Polygonatum multiflorum, lovely hemsleya, Sambucus nigra, Phaseolus lunatus, Centaurium, Macrocystis pyrifera, Turnera diffusa, Anemarrhena asphodeloides, Portulaca pilosa, Humulus lupulus, Coffea arabica, llex paraguariensis, Globularia cordifolia, Oxydendron arboreum, Albizzia julibrissin, Zingiber zerumbet smith, Astragalus membranaceus, Atractylodes macrocephalae, Plantago lanceolata, Leontopodium alpinum (or eldelweiss), Mirabilis jalapa, Apium graveolens, Marrubium vulgare, Buddleja davidii Franch or orchids. The suspension and/or composition according to the invention may be applied locally to the targeted areas.

By way of example, for a facial cosmetic treatment, the European Cosmetics Directive has set a standard application amount for a cream of 2.72 mg/cm²/day/person and for a body lotion of 0.5 mg/cm²/day/person.

According to other particular features, the cosmetic treatment method according to the invention may be combined with one or more other treatment methods aimed at the skin, for instance light, heat or aromatherapy treatments.

According to the invention, it is possible to propose multi-compartment devices or kits intended for performing the method described above, and which could comprise, by way of example, and without this being limiting, in a first compartment a suspension according to the invention, and in a second compartment a complementary active agent, the compositions contained in said first and second compartments being considered here as combination compositions for simultaneous, separate or staggered use over time in particular in one of the treatments defined above.

Preferably, according to the invention, the additional active is chosen from the vitamin B compounds, including the vitamin B3 compounds, including niacinamide, tocopherol, hexamidine, α-lipoic acid, resveratrol or DHEA, hyaluronic acid and retinol, which are active agents very commonly used in topical cosmetic or dermo-pharmaceutical compositions.

According to a fifth aspect, the present invention provides the use of particles according to the invention as defined according to the first aspect, or of a suspension according to the invention as defined above according to the third aspect, or of a composition according to the invention as defined according to the fourth aspect, for oral or topical non-therapeutic cosmetic treatment of the skin and/or its integuments.

The present invention thus covers a non-therapeutic cosmetic treatment method for beautifying or improving the appearance and general condition of the skin and/or integuments and for treating imperfections, in a subject in need thereof, of an effective amount of the suspension according to the invention or of a composition comprising it, in a physiologically acceptable excipient.

The “effective” amount according to the invention, i.e. its dosage in the composition, depends on the intended use of the composition. It depends on various factors, such as the age and condition of the patient and the severity of the disorder. An effective amount means a non-toxic amount sufficient to obtain the desired effect.

These purely cosmetic treatments may be the same as those recommended for peptides already on the market, or treatments newly evidenced. These treatments particularly comprise any type of anti-ageing treatment, for preventing or treating the effects of photo-ageing, particularly fine lines and wrinkles, unsightly spots, signs of skin fatigue such as dark circles and puffiness, slimming treatments, scalp care and/or haircare.

According to a sixth aspect, the present invention provides particles according to the invention according to the first aspect, a suspension according to the invention as defined above according to the third aspect, or a composition according to the invention as defined according to the fourth aspect, for a therapeutic, preferably topical, treatment. Such a treatment may be, for example, a topical anti-inflammatory treatment.

According to a seventh aspect, the present invention proposes the use of particles according to the invention according to the first aspect, of a suspension according to the invention as defined above according to the third aspect, as an active ingredient for manufacturing a physiologically acceptable composition suitable for treating the skin and/or its integuments, orally or topically.

### EXAMPLES

The present invention will be better understood in the light of the detailed description of embodiments given as examples and studies described below.

### A—Preparation of Capsule Suspensions According to the Method of the Invention Starting Materials (all the Weight Percentages are Expressed Relative to the Total Weight of the Mixture Formed by the Various Phases):

Wax: Cetyl palmitate, for example Crodamol® CP (Croda).
Oil: mixture of C8/C10 fatty acid esters, for example Crodamol® GTCC (Croda).
Surfactant: TA1: Lauryl glucoside, for example Plantacare® 1200 UP (BASF);
Peptides: pure in the form of di-, tri-, tetra-, penta-, hexa-peptide powders, optionally with N-terminal acylation, particularly Palmitoylated (Pal) or with N-terminal biotinyl derivatization.
Optionally: a second surfactant TA2, in this case hydrogenated lecithin, for example P75-3™ (Lipoid).
Optionally: a co-solvent for the peptide, such as glycerol or sorbitol.
Optionally: added to one of the phases A to C (see the table below) or via additional phases:
pH adjuster: for example, citric acid in an amount suitable for a pH of 5 to 6;
Bacteriostatic agent: 1% to 6%, for example 1,2-octanediol and/or propanediol;
Antioxidants: 0.01% to 1%, for example tocopherol.

Table 1 below gives examples of formulations according to the invention, followed by the procedure for forming a capsule suspension according to the invention. The values correspond to weight percentages relative to the total weight of the formulation.

TABLE 1

| | | | Biot- | Pal- | Pal- | Pal- | Pal- | Pal- |
|---|---|---|---|---|---|---|---|---|
| | Pal-PP | Pal-GHK | GHK | KMO₂K | GQPR | KTFK | KTTKS | VGVAPG |
| Phase A: | | | | | | | | |
| Wax | 2.5 | 2.5 | 2.5 | 2.5 | 2.5 | 2.5 | 2.5 | 2.5 |
| Oil | 2.5 | / | / | / | 4.5 | 4.5 | 4.5 | / |
| TA1 | 2.5 | / | 2.5 | 2.5 | / | 2.5 | 2.5 | / |
| Phase B: | | | | | | | | |
| Oil | 7.5 | 7.5 | 7.5 | 7.5 | 3 | 3 | 3 | 7.5 |
| TA2 | 0.5 | 0.5 | 0.5 | 0.5 | 0.5 | 0.5 | 0.5 | 0.5 |
| Phase C: | | | | | | | | |
| Water | qs 100 | qs 100 | qs 100 | qs 100 | qs 100 | qs 100 | qs 100 | qs 100 |
| Peptide | 0.01 | 0.01 | 0.01 | 0.01 | 0.01 | 0.01 | 0.01 | 0.024 |
| TA1 | / | 2.5 | 2.5 | / | 2.5 | / | / | 2.5 |
| Co-solvent | / | 10 | / | / | 10 | / | / | / |
| pH adjuster | 0.05 | 0.05 | 0.03 | 0.05 | 0.07 | 0.01 | 0.01 | 0.24 |

### Procedure:

Step 1: parallel preparation of phases (A), (B) and (C); heating where appropriate to a temperature suitable for dissolving the solid starting materials, to make the phases substantially liquid and homogeneous. TA1 (here lauryl glycoside) is either added to phase (A) or phase (C), depending on the solubility of the peptide. The wax-containing phase (A) may contain a part of the oil of phase (B) to help dissolve the peptide if necessary.

When present, the co-solvent is added to the aqueous phase.

Step 2: hot mixing of the three phases (A), (B) and (C), at a temperature at which the three phases remain substantially liquid and homogeneous.

Step 3: hot formation of an emulsion.

Step 4: particle size reduction by hot high-pressure homogenization (three cycles at 500 bar).

Step 5: cooling to room temperature so as to form the submicron particle suspension according to the invention.

### B—Characterization of the Obtained Suspensions

### 1—Methods Used

### 1.1—Macroscopic Characterization:

For each suspension, the presence or absence of heterogeneous aspects, phase separation or precipitation, and colour are visually assessed through a colourless transparent bottle without the use of any instruments. The viscosity is also estimated visually in comparison with water.

1.2—pH:

The pH was measured directly in the formulation (without dilution) using a calibrated potentiometer (pH 4.0 and 7.0).

### 1.3—Particle Size:

Laser diffraction technique (Mastersizer 3000™, Malvern). The suspension is added directly to the water-containing wet dispersion unit until darkening in the 2-8% range. The refractive index used is 1.456, corresponding to the index of wax, i.e. cetyl palmitate. The results are expressed in D50 corresponding to particle diameters at the 50th percentile of the particle size distribution curve, representing the mean particle size.

Polydispersity: the distribution curve also shows particle size homogeneity by means of the index (or range) calculation: (D90-D10)/D50, where D90 and D10 correspond respectively to particle diameters at the 90th percentile and 10th percentile of the particle size distribution curve. A range of less than 2 is considered as a narrow distribution, describing particle size homogeneity.

### 1.4—Encapsulated Peptide Active Content:

This is quantified using an HPLC chromatography method, with a column, mobile phase and detection appropriate to each peptide active agent. An external calibration curve is produced.

Each suspension is treated by liquid/liquid extraction of a mixture composed of isopropanol, a pH 2-2.5 buffer and an organic solvent (acetonitrile or methanol) depending on the peptide to be dosed.

For Pal-KTTKS, Pal-GHK, Pal-GQPR and Pal-VGVAPG peptides, quantification is performed, for example, by HPLC at 35 °C using a Waters Symmetry™ C18 5 μm 4.6×150 mm HPLC column, a mobile phase consisting of an eluent of 99.8/0.1/0.1 v/v/v water/orthosphoric acid/triethylamine and acetonitrile. Detection is performed at 210 nm.

For the Pal-PP peptide, for example, analysis is performed at 40 °C using a Macherey Nagel™ EC 125/4 Nucleodur 100-5 C8 ec column, a mobile phase consisting of a 99.8/0.1/0.1 v/v/v water/orthosphoric acid/triethylamine and acetonitrile eluent. Detection is performed at 210 nm.

For the Biot-GHK peptide, analysis is performed at 40 °C using a Macherey Nagel™ CC 125/4 Lichrospher 100 RP 18 ec 5 μm column, a mobile phase consisting of 25 mM potassium dihydrogen phosphate buffer, pH=2, and 0.01% m/v hexanesulfonic acid ion exchanger in acetonitrile. Detection is performed at 210 nm.

The precision and repeatability of the peptide assay methods allow coverage of greater than 90%.

### 1.5—Zeta Potential and Particle Size by Dynamic Light Scattering (DLS):

The zeta potential is measured using Brookhaven PALS™ equipment on samples of encapsulated peptide or placebo suspension diluted 5000-fold in water. The samples were stored at RT prior to analysis.

The zeta potential (or zeta electrokinetic potential) represents the potential difference between the surface of the particle, covered with opposite, firmly attached ions, and the point of neutrality. It constitutes a good indicator of inter-particle interactions and therefore of colloid stability. A negative zeta potential indicates good “repulsion” between particles in the suspension, preventing particle agglomeration.

### 2—Results

The suspensions obtained according to the method of the invention are homogeneous, with no visible phase separation, a milky white colour and no visible heterogeneity. Their viscosity is low. The results are shown in Table 2 below.

TABLE 2

| | Pal- | Pal- | Biot- | Pal- | Pal- | Pal- | Pal- | Pal- |
|---|---|---|---|---|---|---|---|---|
| | PP | GHK | GHK | KMO₂K | GQPR | KTFK | KTTKS | VGVAPG |
| pH | 8.43 | 5.25 | 4.87 | 5.78 | 5.98 | 4.92 | 5.26 | 5.6 |
| D50 (μm) | / | 0.239 | 0.316 | 0.261 | 0.229 | 0.343 | 0.254 | 0.304 |
| Polydispersity | / | 1.31 | 1.55 | 1.27 | 1.33 | 1.15 | 1.29 | 0.27 |
| index | | | | | | | | |
| Dosage | 100 | 105 | 107 | 78 | 96 | 61 | 100 | 260 |
| | ppm | ppm | ppm | ppm | ppm | ppm | ppm | ppm |

The zeta potential was measured negative on a suspension of encapsulated Pal-GHK. The results presented above show that by using similar formulations in the frame of the present invention, it was possible to encapsulate different peptides, ranging in composition, number and amino acid sequence. In all cases, the formulations were obtained with the desired particle size, polydispersity and degree of encapsulation, and were approved in the Day 0 analysis. Samples were then produced for study during stability at room temperature (RT) and at 40 °C.

### C—Proof of Encapsulation and Half-Life: Trypsin Digestion

### 1—Principle and Protocol:

The study was performed on a suspension of Pal-KTTKS encapsulated according to the invention, using trypsin, an enzyme that hydrolyses a peptide whose sequence contains the amino acid lysine. The peptide is placed in the presence of trypsin and the amount of peptide remaining, i.e. not hydrolysed by trypsin, is measured as a function of time. Only the peptide released from the capsule over time is susceptible to hydrolysis.

At T0, measurements make it possible to confirm that the peptide is encapsulated.

The kinetics will then make it possible to determine the half-life, which constitutes an indicator of the peptide's accessibility to the enzyme, and therefore an indicator of the peptide's protection by the capsule.

Two solutions are compared: one containing the encapsulated peptide (100 ppm) and the other the free (i.e. non-encapsulated) peptide, also at 100 ppm. Acetate buffer solution at pH=5 containing 0.003% trypsin is added. The samples are heated to 25 °C. The enzymatic reaction is stopped to perform the measurements by adding isopropyl alcohol. Peptide quantification is performed by HPLC using the method described in paragraph B 1-4 above.

### 2—Results:

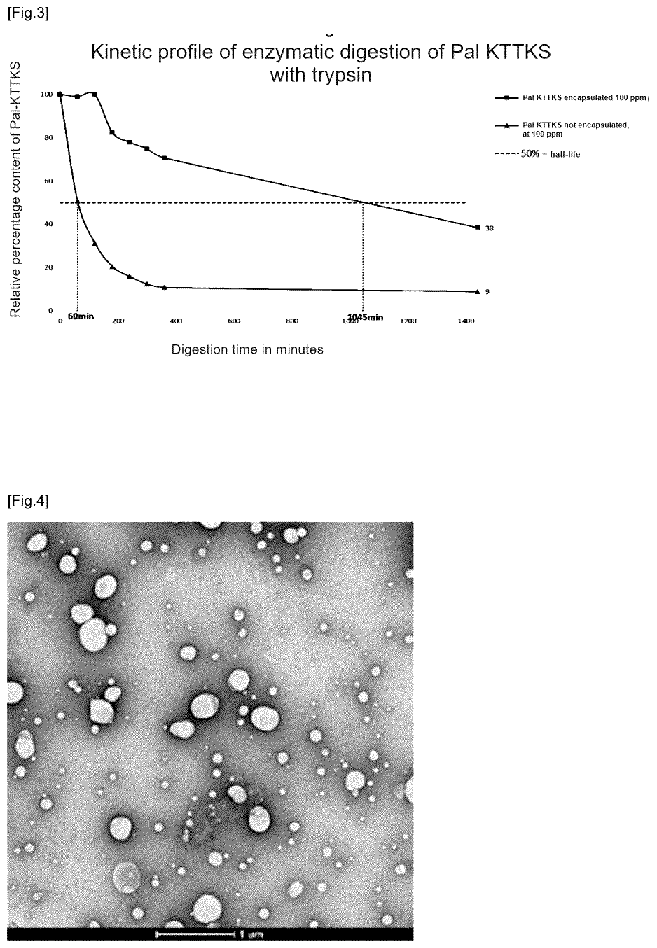

FIG. 3 is a graph showing the Pal-KTTKS content (%) as a function of time (min) at 25 °C. In this figure, it is seen that the Pal-KTTKS content of the encapsulated Pal-KTTKS is reduced by 50% in 1045 minutes, whereas for free Pal-KTTKS the content decreases by 50% in 60 minutes. It may thus be concluded that Pal-KTTKS is effectively protected in the capsules, since it does not follow the same curve as free, non-encapsulated Pal-KTTKS.

Hydrolysis monitoring is performed under the chromatographic analysis conditions described in paragraph B 1-4 above. The chromatogram initially shows a chromatographic peak of Pal-KTTKS at 10.5 min, and then as enzymatic degradation proceeds, this peak reduces and two new peaks are seen, corresponding to Pal-KTTK at 10.26 min and Pal-K at 13 min.

The half-life values T1/2 (i.e. loss of 50% of the initial content) are given in Table 3 below:

TABLE 3

| | T1/2 (min) |
|---|---|
| | from the |
| | graph of |
| | FIG. 3 |
| Encapsulated Pal-KTTKS | 1045 |
| Free Pal-KTTKS | 60 |

These results show that the half-life of encapsulated Pal-KTTKS is more than fifteen times longer than that of free Pal-KTTKS. A longer half-life will confer greater stability to the peptide in the skin because its degradation on contact with the skin enzymes will be slower.

A greater amount of intact peptide will be able to be transported to the site of activity.

### D—Encapsulation Efficacy Demonstrated by Tangential Flow Filtration Analysis

### 1—Principle and Protocol:

Tangential flow filtration, also known as cross-flow filtration, allows suspended particles to be separated by passing the suspension along the surface of a membrane. The Sartorius Vivaflow™ 50 R filtration membrane, with a cutoff threshold of 100 kDa MWCO, allows free peptide to pass through the membrane, on the one hand, and capsule retention, on the other hand. The liquid phase permeates through the membrane due to a pressure difference across the membrane. After filtration, a retentate (non-permeated phase containing the particles) and a filtrate (permeated aqueous phase) are recovered.

After cross-filtration, the free (non-encapsulated) peptide ends up in the filtrate, while the encapsulated peptide cannot cross the membrane and ends up in the retentate.

To determine the encapsulation efficacy, two comparative tests are performed: tangential filtration of a peptide suspension according to the invention and a placebo aqueous solution of free peptide at the same concentration as the encapsulated peptide. The amount of free peptide in the various fractions, i.e. filtrate and retentate (including losses in the system), is measured by HPLC, using the HPLC method described in paragraph B 1-4 above.

### 2—Results:

The test was performed on a Pal-KTTKS suspension according to the invention and on a Pal-GHK suspension according to the invention.

TABLE 4

| | | Relative | Relative |
|---|---|---|---|
| | | content of | content of |
| | Analysed | Pal-KTTKS | Pal-GHK |
| | fraction | (%) | (%) |
| Peptide | filtrate | 98 | 95 |
| Free form | retentate | 5 | 6 |
| Peptide | filtrate | <LOD | 7 |
| Encapsulated form | retentate | 74 | 74 |

The results, expressed as a percentage of the amount used, show:

For free Pal-KTTKS,
98% of the Pal-KTTKS passes through the membrane and is assayed in the filtrate.

For encapsulated Pal-KTTKS,
No Pal-KTTKS was found in the filtrate at the limit of detection (LOD (~2 ppm)),
By adding the part lost on the system to the retentate assay, the yield obtained is more than 74% of Pal-KTTKS transported in encapsulated form, which has not passed through the membrane.

Similarly, for Pal-GHK, the results show:

For free Pal-GHK,
95% of the Pal-GHK passes through the membrane and is assayed in the filtrate.

For encapsulated Pal-GHK,
About 7% Pal-GHK is found in the filtrate,
By adding the part lost on the system to the retentate assay, the yield obtained is more than 74% of Pal-GHK transported in encapsulated form, which has not passed through the membrane.

This experiment demonstrates the efficacy of the encapsulation: 88% of the Pal-KTTKS in the suspension according to the invention is indeed found inside the capsules.

### E—Particle Morphology:

### 1—Differential Scanning Calorimetry (DSC) Analysis

An analysis was performed at 10 °C/min from 0 to 180 °C on a suspension of Pal-KTTKS encapsulated according to the invention. The thermogram is shown in FIG. 2, with a single large endothermic peak at about 50.09 °C combined attributed to the mixture of wax and surfactants. This analysis confirms that these are waxy particles, which are solid at room temperature, inside which the peptide active agent is encapsulated.

### 2—Transmission Electron Microscopy (TEM) Analysis:

A suspension diluted to 5% in water was applied to a support and dried for 1 min. The sample was then coloured with 2% uranyl acetate for 1 min.

An image of a sample of encapsulated Pal-KTTKS suspension is shown in FIG. 4, showing regular-shaped, substantially spherical particles. The TA1 coating (lauryl glucoside in this example) is visible around the sphere. The particle size is also compatible with the laser diffraction analyses presented above.

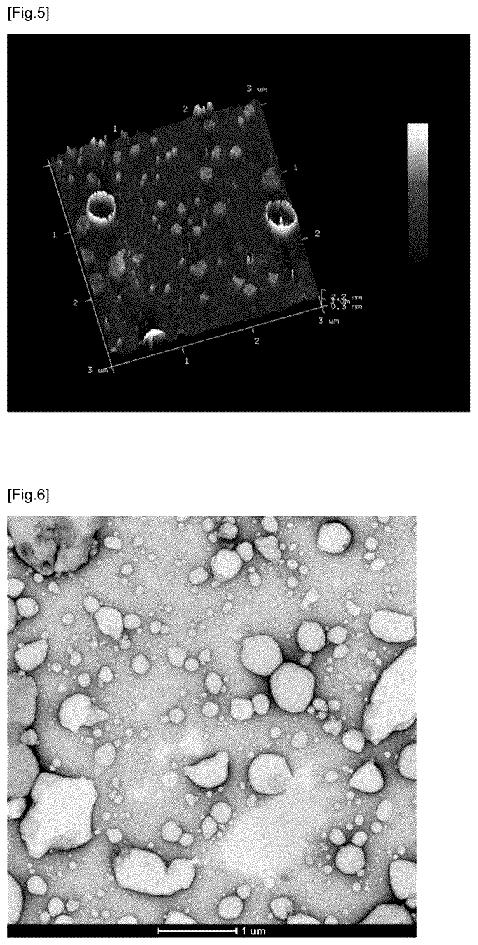

An image of a sample of encapsulated Pal-GHK suspension is shown in FIG. 6. It can also be seen that the particles have a regular shape and are substantially spherical. The TA1 coating is also visible around the sphere. The particle size is also compatible with the laser diffraction analyses presented above.

### 3—Atomic Force Microscopy (AFM) Analysis

AFM allows analysis of a surface point by point, by means of a scanning probe consisting of a fine tip. It allows observation of objects on a very small scale.

FIG. 5 shows an AFM image of a sample of encapsulated Pal-KTTKS suspension according to the invention (at 100 ppm). The image shows that the particles have been split by the probe, demonstrating that they have solid thin shell but remain flexible and deformable. The core-shell structure of the particles is visible.

### F—Stability Studies

For these studies, encapsulated peptide formulations were stored in HDPE polyethylene bottles at the study temperature (room temperature and 40 °C). At each analysis time, a new 30 g bottle is opened for the first time. All the samples are handled under regular laboratory lighting and normal air conditions. No inert gas is used to fill the bottles or to handle the samples.

### 1—Stability at Room Temperature

The following encapsulated peptides were monitored: Pal-KTTKS, Pal-GHK, Pal-GQPR, Pal-KTFK, Pal-KMO₂K, Pal-VGVAPG and Pal-PP.

### 1.1—Results for Encapsulated Pal-KTTKS:

TABLE 5

| | Time | | | | |
|---|---|---|---|---|---|
| Analysis | 0 | 1 month | 3 months | 6 months | 12 months |
| pH | 5.26 | 5.42 | 5.41 | 5.41 | 5.98 |
| D50 (μm) | 0.254 | 0.263 | 0.280 | 0.292 | 0.276 |
| Dosage (ppm) | 100 | 89 | 81 | 85 | 89 |

### 1.2—Results for Encapsulated Pal-GHK:

TABLE 6

| | Time | | |
|---|---|---|---|
| Analysis | 0 | 1 month | 3 months |
| pH | 5.55 | 5.62 | 5.63 |
| D50 (μm) | 0.298 | 0.385 | 0.424 |
| Dosage (ppm) | 105 | 107 | 72 |

### 1.3—Results for Encapsulated Pal-GQPR

TABLE 7

| | Time | | |
|---|---|---|---|
| Analysis | 0 | 1 month | 3 months |
| pH | 5.98 | 5.92 | 6.21 |
| D50 (μm) | 0.229 | 0.262 | 0.287 |
| Dosage (ppm) | 112 | 106 | 104 |

### 1.4—Results for Encapsulated Pal-KTFK

TABLE 8

| | Time | | | |
|---|---|---|---|---|
| Analysis | 0 | 1 month | 3 months | 6 months |
| pH | 4.92 | 4.95 | 5.09 | 4.96 |
| D50 (μm) | 0.343 | 0.283 | 0.366 | 0.370 |
| Dosage (ppm) | 61 | 77 | 58 | 75 |

### 1.5—Results for Encapsulated Pal-KMO₂K

TABLE 9

| | Time | |
|---|---|---|
| Analysis | 0 | 2 months |
| pH | 5.65 | 5.59 |
| D50 (μm) | 0.263 | 0.275 |
| Dosage (ppm) | 75.7 | 78 |

### 1.6—Results for Encapsulated Pal-VGVAPG

TABLE 10

| | Time | |
|---|---|---|
| Analysis | 0 | 3 months |
| pH | 5.6 | 5.9 |
| D50 (μm) | 0.304 | 0.295 |
| Dosage (ppm) | 240 | 238 |

### 1.7—Results for Encapsulated Pal-PP

TABLE 11

| | Time | | |
|---|---|---|---|
| Analysis | 0 | 1 month | 2 months |
| pH | 8.43 | 8.22 | / |
| D50 (μm) | / | / | / |
| Dosage (ppm) | 100 | 106 | 102 |

All these results show good room-temperature stability of the suspension according to the invention, with pH, mean capsule size, polydispersity and also encapsulated peptide content changing slightly but within acceptable limits. There is no indication of capsule degradation or destabilization.

For Pal-KTTKS, stability of up to 1 year at room temperature was observed. The formulation is stable, capable of maintaining capsule size and the amount of encapsulated peptide. For comparative purposes, Matrixyl® containing non-encapsulated Pal-KTTKS must be stored at about 5 °C.

### 2—Accelerated Stability

The study was performed at 40 °C. At this temperature, degradation is accelerated. There is a correlation with degradation at room temperature, 90 days at 40 °C being equivalent to 1 year at room temperature. This study thus constitutes a good indicator of the stability of the encapsulated peptide suspensions according to the invention.

The following encapsulated peptides were monitored: Pal-KTTKS, Pal-GHK, Pal-GQPR, Pal-KTFK and Pal-PP.

### 2.1—Results for Encapsulated Pal-KTTKS

TABLE 12

| | Time and conditions | | |
|---|---|---|---|
| | | 30 days | 90 days |
| Analysis | 0 | at 40 °C | at 40 °C |
| pH | 5.26 | 5.43 | 5.42 |
| D50 (μm) | 0.254 | 0.268 | 0.279 |
| Assay (ppm) | 100 | 100 | 90 |

### 2.2—Results for Encapsulated Pal-GHK

TABLE 13

| | Time and conditions | | |
|---|---|---|---|
| | | 30 days | 90 days |
| Analysis | 0 | at 40 °C | at 40 °C |
| pH | 5.25 | 5.21 | 5.24 |
| D50 (μm) | 0.239 | 0.237 | 0.245 |
| Assay (ppm) | 105 | 109 | 72 |

### 2.3—Results for Encapsulated Pal-GQPR

TABLE 14

| | Time and conditions | | |
|---|---|---|---|
| | | 30 days | 90 days |
| Analysis | 0 | at 40 °C | at 40 °C |
| pH | 5.98 | 6.14 | 6.00 |
| D50 (μm) | 0.229 | 0.25 | 0.283 |
| Assay (ppm) | 112 | 112 | 92 |

### 2.4—Results for Encapsulated Pal-KTFK

TABLE 15

| | Time and conditions | |
|---|---|---|
| | | 90 days |
| Analysis | 0 | at 40 °C |
| pH | 4.92 | 4.97 |
| D50 (μm) | 0.343 | 0.366 |
| Assay (ppm) | 61 | 76 |

### 2.5—Results for Encapsulated Pal-PP

TABLE 16

| | Time and conditions | |
|---|---|---|
| | | 60 days |
| Analysis | 0 | at 40 °C |
| Assay (ppm) | 100 | 102 |

All these results show that encapsulated peptides, even of different peptide sequences, are stable at room temperature for at least 3 months, and for Pal-KTTKS up to 1 year, and at least 3 months in accelerated stability at 40 °C.

### G—Release Profile

The mechanism of release through a combination of diffusion and erosion was studied. Diffusion through the capsule wax occurs naturally between areas of high and low concentration. When the encapsulated peptide active is applied to the skin, a concentration gradient is formed, from which the diffusion method begins.

To demonstrate the diffusion method, a sample of Pal-GHK encapsulated according to the invention at 100 and 1000 ppm is placed in a dialysis bag sealed with clips. The dialysis bag has a smaller pore size than the capsule size, the capsules being consequently retained in the bag. The bag is placed in a control medium (water acidified with 0.066% citric acid with 3% Tween 80, and 30% EtOH added to this solution) and agitated at constant temperature.

This agitation simulates general wear and erosion causing the release of the encapsulated contents, which then pass through the pores of the dialysis bag into the control medium.

Samples of the medium are collected over time and analysed by HPLC to confirm the presence and content of released Pal-GHK. This test demonstrates the ability of particles according to the invention to deliver the encapsulated peptide active agent to the skin over an extended period of time.

Erosion occurs during application to the skin or its integuments, when the outer layer of the capsules begins to break down, releasing the peptide active. Diffusion and erosion occur simultaneously, accelerating the release of the active. The rate of diffusion ultimately reduces as the concentration gradient decreases, and the peptide active will then only be released by erosion.

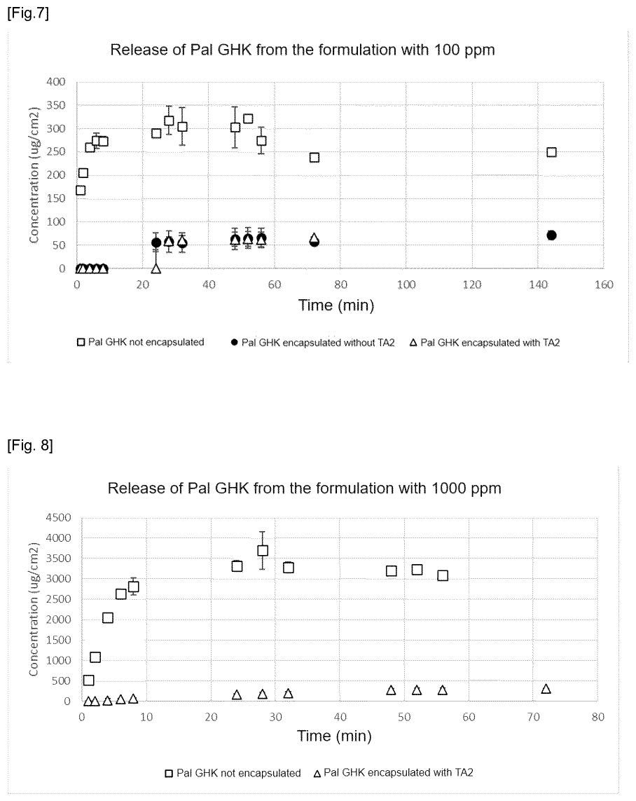

FIG. 8 and FIG. 9 show the release profiles over time for Pal-GHK at 100 and 1000 ppm, respectively, compared with the free peptide.

As can be seen from these figures, the Pal-GHK contained in the capsule is released at a slower rate than the free (non-encapsulated) peptide. This makes it possible to conclude that release is better controlled over time when the peptide is encapsulated according to the invention compared to the free, non-encapsulated peptide, thus demonstrating the benefit of the encapsulation according to the invention and the ability to continuously deliver a benefit linked to the biological activity of the peptide active.

FIG. 8 shows the release of encapsulated Pal GHK without and with the second surfactant, for example hydrogenated lecithin. The figure shows that the addition of the second surfactant does not change the profile and is preferred for the formulation for particle stability.

### H—Comparative Studies

1—TA1 Surfactant of Alkyl Glucoside of at Least One Saturated C10-C16 Fatty alcohol: Comparison of Lauryl Glucoside and Decyl Glucoside (Maintaining all the Other parameters)

TABLE 17

| | Pal-KTTKS | | Pal-GHK | |
|---|---|---|---|---|
| | Formation of a | | Formation of a | |
| | suspension at | | suspension of | |
| | T0 of submicron | | submicron | |
| | particles | Stability over | sized particles | Stability over |
| | (D50 <1 μm) | time at RT | at T0 | time at RT |
| Lauryl | Yes | Yes (12 months) | Yes | Yes (3 months) |
| glucoside | pH = 5.26 | pH = 5.98 | pH = 5.20 | pH = 6.11 |
| | D50 (μm) = 0.254 | D50 (μm) = 0.276 | D50 (μm) = | D50 (μm) = 0.243 |
| | Encapsulated | Encapsulated | 0.250 | Encapsulated |
| | peptide = 100 ppm | peptide = 89 ppm | Encapsulated | peptide = 100 ppm |
| | | | peptide = 77 | |
| | | | ppm | |
| Decyl | Yes | Yes (3 months) | Yes | Yes (3 months) |
| glucoside | pH = 4.87 | pH = 4.94 | pH = 4.98 | pH = 5.08 |
| | D50 (μm) = 0.325 | D50 (μm) = 0.363 | D50 (μm) = | D50 (μm) = 0.319 |
| | Encapsulated | Encapsulated | 0.274 | Encapsulated |
| | peptide = 69 ppm | peptide = 83 ppm | Encapsulated | peptide = 78 ppm |
| | | | peptide = 91.5 | |
| | | | ppm | |

The two surfactants also provide a particle suspension that is acceptable in terms of particle size (D50<0.6 μm) and stability over time (at least 3 months).

2—Surfactant TA1 of polyglycerol ester of saturated C10-C16 fatty acid and of ethoxylated Polysorbate: results compared between lauryl glucoside, a mixture of polyglyceryl-4 laurate/sebacate and polyglyceryl-6 caprylate/caprate (Natragem S140 from the company Croda) and Tween 80 (Polysorbate 80) (maintaining all the other parameters).

TABLE 18

| | Pal-KTTKS |
|---|---|
| | Formation of a homogeneous suspension |
| | at T0 of submicron particles (D50 <1 μm) |
| Lauryl glucoside | Yes |
| | pH = 5.26 |
| | D50 (μm) = 0.254 |
| | Encapsulated peptide = 100 ppm |
| Natragem S140 | Yes |
| (1.75% relative to | pH = 4.12 |
| the total weight of | D50 (μm) = 0.317 |
| the formulation) | Encapsulated peptide = 110 ppm |
| Tween 80 | Yes |
| (1.75% relative to | pH = 5.03 |
| the total weight of | D50 (μm) = 0.259 |
| the formulation) | Encapsulated peptide = 99 ppm |

Natragen S140 and Tween 80 are suitable surfactants according to the invention, for obtaining a particle suspension that is acceptable in terms of particle size (D50<0.6 μm) and polydispersity.

### 3—Addition of Surfactant TA2 (in the Example Hydrogenated Lecithin)

TABLE 19
Tests with Pal-GHK

| | | Formation of a | | |
|---|---|---|---|---|
| | | homogeneous, stable, | | |
| | | phase separation-free | | |
| | | suspension of | | Stability over time |
| | TA2 | submicron particles, | | at 3 months at |
| TA1 | Lipoid | with no visible | Stability over | 40 °C (equivalent |
| HLB >10 | P75-3 | particles, at T0 | time at 3 months at RT | to 12 months at RT) |
| X | | Yes | Yes | No: Phase |
| | | pH = 4.93 | pH = 4.93 | separation |
| | | D50 (μm) = 0.264 | D50 (μm) = 0.353 | pH = 5 |
| | | Encapsulated | Encapsulated | D50 (μm) = 5.0 |
| | | peptide = 74 ppm | peptide = 100 ppm | Encapsulated |
| | | | | peptide = no |
| | | | | possible dosage |
| X | X | Yes | Yes | Yes |
| | | pH = 5.29 | pH = 6.06 | pH = 5.24 |
| | | D50 (μm) = 0.239 | D50 (μm) = 0.247 | D50 (μm) = 0.245 |
| | | Encapsulated | Encapsulated | Encapsulated |
| | | peptide = 74 ppm | peptide = 101 ppm | peptide = 71 ppm |

By adding the second surfactant with an HLB<6 (hydrogenated lecithin in the example), the size of the submicron particles is maintained over time. At 3 months at RT, the particle size is still acceptable without TA2 in the formula, and at the same time at 40 °C, equivalent to 12 months at RT, the size is well over 1 micron and the dispersion is heterogeneous (two phases). The results show that for particle formation and stability at room temperature, a single TA1 surfactant is sufficient. However, for stability at temperatures higher than room temperature, the second surfactant TA2 is recommended.

### 4—Peptide Active Content

TABLE 20
Test with Pal-GHK

| | Formation of | |
|---|---|---|
| | a suspension | |
| | of submicron | Stability over time at RT, |
| | particles at T0 | at 1 month |
| 100 ppm | Yes | Yes |
| | D50 (μm) = 0.318 | D50 (μm) = 0.382 |
| 500 ppm | Yes | Yes |
| | D50 (μm) = 0.324 | D50 (μm) = 0.341 |
| 1000 ppm | Yes | Yes |
| | D50 (μm) = 0.448 | D50 (μm) = 0.452 |

### 5—Choice of the Wax

TABLE 21
Test with Pal-KTTKS

| | Formation of |
|---|---|
| | a suspension |
| | of submicron |
| | particles at T0 |
| Cetyl palmitate | Yes |
| | pH = 5.15 |
| | D50 (μm) = 0.27 |
| Triglyceride esters of | Yes |
| C18-C36 fatty acids | pH = 5.61 |
| (Syncrowax ®) | D50 (μm) = 0.392 |

Both types of wax are suitable for use according to the invention. Cetyl palmitate-type waxes are preferred as they allow the polydispersity to be reduced.

### 6—Amount of Wax

TABLE 22
Test with Pal-KTTKS

| | Formation of | |
|---|---|---|
| | a suspension | |
| | of submicron | Stability over time at RT |
| | particles at T0 | (6 months) |
| 2.5% wax | Yes | Yes |
| | PH 5.26 | pH 5.41 |
| | D50 (μm) = 0.254 | D50 (μm) = 0.292 |
| 5% wax | Yes | Yes |
| | pH 5.45 | pH 5.74 |
| | D50 (μm) = 0.307 | D50 (μm) = 0.342 |

### 7—Adding a Co-Solvent

TABLE 23
Test with Pal-GHK

| | Formation of | |
|---|---|---|
| | a suspension | |
| | of submicron | Stability over time |
| | particles at T0 | 1 month |
| Without glycerol | Yes | Yes |
| | pH 5.25 | pH 6.11 |
| | D50 (μm) = 0.239 | D50 (μm) = 0.243 |
| 10% glycerol | Yes | Yes |
| | pH 4.88 | pH 4.90 |
| | D50 (μm) = 0.232 | D50 (μm) = 0.276 |

### 8—Oil Type

TABLE 24
Test with Pal-GHK

| | Formation of a suspension |
|---|---|
| | of submicron particles at T0 |
| Caprylic/capric | Yes |
| triglycerides | pH = 4.73 |
| | D50 (μm) = 0.290 |
| | Encapsulated peptide = 99 ppm |
| Ethyl oleate | Yes |
| | pH = 6.08 |
| | D50 (μm) = 0.300 |
| | Encapsulated peptide = 119 ppm |
| Triisostearin | Yes |
| (isostearic acid | pH = 6.01 |
| triglyceride) | D50 (μm) = 0.373 |
| | Encapsulated peptide = 103 ppm |

### 9—Oil Quantity

TABLE 25
Test with encapsulated Pal-KTTKS and Crodamol GTCC

| | Formation of a suspension |
|---|---|
| | of submicron particles at T0 |
| 7.5 % Crodamol | Yes |
| GTCC | D50 (μm) = 0.290 |
| | D90 (μm) = 0.521 |
| 15% Crodamol | Yes |
| GTCC | D50 (μm) = 0.345 |
| | D90 (μm) = 0.563 |

Increasing oil content led to a slightly increase in particle size, which was expected due to higher oil core concentration. Both formulations presented similar characteristics and stability after centrifugation.

10—Peptides with Different Derivatization

TABLE 26
Test encapsulating peptides at 100 ppm with different derivatization

| | Formation of | |
|---|---|---|
| | a suspension | |
| | of submicron | Stability over time |
| | particles at T0 | 1 month at 40 °C |
| H-KTTKS (no derivatization, | Yes | Yes |
| SEQ ID NO. 8) | pH 5.63 | pH 5.90 |
| | D50 (μm) = 0.230 | D50 (μm) = 0.229 |
| Pal-KTTKS | Yes | Yes |
| | pH 5.00 | pH 5.13 |
| | D50 (μm) = 0.292 | D50 (μm) = 0.302 |
| N-Acetyl-YR-O-hexadecyl | Yes | Yes |
| ester | pH 8.70 | pH 8.70 |
| | D50 (μm) = 0.234 | D50 (μm) = 0.237 |
| Lauroyl-KTSKS | Yes | Yes |
| (SEQ ID NO. 9) | pH 8.72 | pH 8.88 |
| | D50 (μm) = 0.316 | D50 (μm) = 0.271 |
| Pentynoic-KTTKS | Yes | Yes |
| (SEQ ID NO. 10) | pH 8.75 | pH 8.88 |
| | D50 (μm) = 0.238 | D50 (μm) = 0.238 |
| Octanoyl-IW | Yes | Yes |
| | pH 9.38 | pH 8.88 |
| | D50 (μm) = 0.423 | D50 (μm) = 0.423 |

After preparation, the formulations were stable, homogeneous, white, and opaque. No visible phase separation was seen as well as no separation after centrifugation (2000 rpm, 20 min).

It was possible to use peptides with no derivatization and different lengths and characteristics of lipid chains in the formulations.

All samples demonstrated stability for 30 days, independently of the peptide. Particle size differs due to peptide presence in the capsules, but all leading to stable homogeneous products.

I—Example of Encapsulation of a Peptide According to the Invention and adsorption of a Second Peptide

To the obtained suspension of encapsulated Pal-KTTKS particles (obtained from the formulation described in Table 25 above, containing 100 ppm of Pal-KTTKS), once cooled to room temperature, 100 ppm of a second peptide, Pal-KTFK, pre-dissolved in water, are added and the mixture is stirred. A suspension of particles is obtained, on the outside of which the Pal-KTFK is adsorbed (release test shown below). The assay of the two peptides is given in the table below.

TABLE 27
Encapsulated Pal-KTTKS and adsorbed Pal-KTFK

| Analysis | Peptide | 3 months at 40 °C |
|---|---|---|
| Dosage (ppm) | Pal-KTTKS | 100 |
| | Pal-KTFK | 78 |

The same tangential filtration experiment as described in paragraph D-1 was performed to show that the second peptide, Pal-KTFK, is adsorbed onto the particles and is not found in free form in the suspension.

The results obtained are described in the following table:

TABLE 28

| | | Relative |
|---|---|---|
| | | content of |
| | Fraction | Pal-KTFK |
| | analysed | (%) |
| Adsorbed Pal-KTFK | Filtrate | 1.2 |
| | Retentate | 82.5 |

The results, expressed as a percentage of the amount used, show that:
1.2% of Pal-KTFK is found in the filtrate;
nearly 82.0% of the Pal-KTFK is thus adsorbed by the particles and can be advantageously “carried” by the particles together with the encapsulated Pal-KTTKS.

### J—Example of Encapsulation of a Peptide Mixture According to the Invention (Co-Encapsulation)

Starting materials (all the weight percentages are expressed relative to the total weight of the mixture formed by the various phases):
Wax: cetyl palmitate, Crodamol® CP (Croda),
Oil: mixture of C8/C10 fatty acid esters, Crodamol® GTCC (Croda),
Surfactant TA1: Lauryl glucoside (Plantacare® 1200 UP, BASF),
Surfactant TA2: Hydrogenated lecithin (P75-3, Lipoid),
Peptide mixture: Pal-GHK and Pal-GQPR in pure form (powders),
Co-solvent: glycerol,
pH adjuster: citric acid,

Table 29 below gives the details of the formulation.

TABLE 29

| | m/m % |
|---|---|
| Phase A: | |
| Wax | 2.5 |
| TA1 | / |
| Phase B: | |
| Oil | 7.5 |
| TA2 | 0.5 |
| Phase C: | |
| Water | qs 100 |
| Peptide Pal-GHK | 0.01 |
| Peptide Pal-GQPR | 0.01 |
| TA1 | 2.5 |
| Co-solvent | 5 |
| pH adjuster | 0.05 |

### Procedure:

Step 1: parallel preparation of phases (A), (B) and (C); heating to a temperature suitable for dissolving the solid starting materials to make the phases substantially liquid and homogeneous.

Step 2: hot mixing of the three phases (A), (B) and (C), at a temperature at which the three phases remain substantially liquid and homogeneous.

Step 3: hot forming an emulsion.

Step 4: particle size reduction by hot high-pressure homogenization (three cycles at 500 bar).

Step 5: cooling to room temperature so as to form the suspension of submicron capsules according to the invention.

The following results are obtained:

TABLE 30
Co-encapsulation of Pal-GHK and Pal-GQPR

| Formation of a | |
|---|---|
| homogeneous suspension | Stability over time, |
| of submicron particles at T0 | 1 month at RT |
| Yes | Yes |
| pH 5.58 | pH 5.65 |
| D50 (μm) = 0.268 | D50 (μm) = 0.321 |
| Pal-GHK assayed at 83 ppm | Pal-GHK assayed at 78 ppm |
| Pal-GQPR assayed at 79 ppm | Pal-GQPR assayed at 79 ppm |

These results show that the method according to the invention allows a mixture of two peptides to be encapsulated in a stable manner over time.

### K—Examples of Finished Product Formulations

Various formulations can be developed, comprising as active ingredient a suitable weight percentage of a suspension according to the invention encapsulating a peptide active, as prepared according to the examples described above.

These formulations may contain additional active ingredients, where appropriate to support and/or complement the activity of the active ingredient according to the invention. These ingredients may be of any category depending on their function(s), the place of application (body, face, neck, bust, hands, scalp, hair, bodily hairs, etc.), the desired end effect and the targeted consumer, for example anti-ageing, moisturizing, firming, anti-redness, anti-stretchmark, sunscreen, pigmenting, etc.

### Formulation Example 1: Cream Form

TABLE 31

| | | Ingredient |
|---|---|---|
| Starting materials | INCI name | (w/w %) |
| Phase A | | |
| H₂O | Water | qs 100 |
| Carbopol Ultrez 10 ™ | Carbomer | 0.30 |
| Phase B | | |
| Brij S2-SS-(RB) ™ | Steareth-2 | 0.40 |
| Brij S10-SO-(RB) ™ | Steareth-10 | 1.20 |
| Crodafos CES-PA-(RB) ™ | Cetearyl Alcohol | 4.00 |
| | (and) Dicetyl | |
| | Phosphate (and) | |
| | Ceteth-10 Phosphate | |
| Crodacol CS90-PA-(RB) ™ | Cetearyl Alcohol | 1.50 |
| Crodamol AB-LQ-(RB) | C12-15 Alkyl | 1.50 |
| | Benzoate | |
| Crodamol OSU-LQ-(JP) ™ | Diethylhexyl | 7.00 |
| | Succinate | |
| Phase C | | |
| Glycerol | Glycerin | 2.50 |
| Octanediol | Caprylyl Glycol | 0.50 |
| Phase D | | |
| Phenoxyethanol | Phenoxyethanol | qs |
| Phase E | | |
| Potassium sorbate | Potassium Sorbate | sb |
| Phase F | | |
| H₂O | Water | 5.00 |
| 30% NaOH | Sodium Hydroxide | 0.75 |
| Phase G | | |
| Suspension according to the invention | — | 3.00 |

Procedure: Swell phase A without stirring for 30 min and heat to 75 °C on a water bath. Heat phase B to 75 °C on a water bath, mixing well. Melt phase C and mix. Mix phase D with pre-cooled phase C. Pour phase C + D into phase A, with stirring using a rotor-stator mixer, v = 500 rpm. Homogenize well. Add phase B to the preceding phase, with stirring using a rotor-stator mixer, v = 1000 rpm. Add phase E extemporaneously to the preceding phase, with stirring using a rotor-stator mixer, v = 1000 rpm. Homogenize well. Add phase F and homogenize well. Below 40 °C, add phase G and homogenize well.

Examples of ingredients that can be added to this formulation (sold by Sederma): a moisturizing ingredient such as OptimHyal® and/or an ingredient for protecting against the harmful effects of blue light, such as Synchrolife®.

### Formulation Example 2: Shampoo Form

TABLE 32

| Starting materials | INCI name | Function | w/w % |
|---|---|---|---|
| Phase A | | | |
| Empicol ® ESB3 | Sodium Laureth Sulfate | Surfactant | 20 |
| Crodateric ™ CAB 30 | Aqua (and) | Surfactant | 15 |
| | Cocamidopropyl Betaine | | |
| Incromine ™ Oxide C | Cocamidopropylamine | Surfactant | 3 |
| | Oxide (and) Aqua | | |
| Crovol ™ A70 | PEG-60 Almond | Solubilizing | 3 |
| | Glycerides | agent | |
| Phase B | | | |
| Water | Aqua | — | qs 100 |
| Preservative | — | Preservative | qs |
| Sodium chloride | Sodium Chloride | pH adjuster | 2.00 |
| Phase C | | | |
| Suspension according | — | Active agent | 3.00 |
| to the invention | | | |

Procedure: Prepare phases A and B thoroughly homogenized. Pour phase A into phase B with stirring. Homogenize carefully. Add phase C. Mix thoroughly.

1-30. (canceled)

31. Submicron particle comprising a peptide active consisting of a peptide or a peptide mixture and/or an amino acid or an amino acid mixture, wherein the particle is substantially spherical, consisting of an oily core containing the peptide active surrounded by a shell of wax that is solid at room temperature, at least one non-ionic surfactant with an HLB substantially greater than or equal to 10 forming an outer layer on the wax shell.

32. The particle according to claim 30, comprising a second peptide active adsorbed onto the outer layer.

33. The particle according to claim 30, wherein a ratio of weight percentage of wax to weight percentage of oil is between 1:1 and 1:10.

34. The particle according to claim 30, wherein the first surfactant (TA1) is chosen from at least one alkylglucoside or alkylpolyglucoside of at least one saturated C10-C16 fatty alcohol, and/or at least one polyglycerol ester of saturated C10-C16 fatty acid, and/or at least one polysorbate, poloxamer and/or polyvinyl derivative.

35. The particle according to claim 30, characterized by a second surfactant (TA2) with an HLB substantially less than or equal to 6.

36. The particle according to claim 35, wherein the second surfactant (TA2) is a phospholipid chosen from phosphatidic acid, a phosphatidylcholine or lysophosphatidylcholine, glycerophosphocholine, phosphatidylserine or lysophosphatidylserine, phosphatidylethanolamine, phosphatidylinositol or sphingomyelin.

37. The particle according to claim 30, wherein the wax is chosen from cetyl palmitate, glyceryl tribehenate, glyceryl stearate or tristearate or a wax of plant or animal origin.

38. The particle according to claim 30, wherein the oil is chosen from essentially C8-C18 fatty acids, mono-, di- or tri-esters of essentially C8-C18 fatty acid(s), comprising mono-, di- or tri-glycerides of fatty acids, and/or fatty alcohols comprising C8-C18 carbon chains.

39. The particle according to claim 30, wherein the peptide active corresponds to the following general formula (A):
X-(Xaa)n-Z
wherein:
Xaa is an amino acid, derivative or analogue;
n is an integer ≥1, when n≥2 the amino acids being chosen independently of each other;
N-terminal X chosen from H, —CO—R1, —SO2-R1 or a biotinoyl group;
C-terminal Z chosen from OH, OR1, NH2, NHR1 or NR1R2; and
R1 and R2 being, independently of each other, chosen from an alkyl, aryl, aralkyl, alkylaryl, alkoxy, saccharide and aryloxy group, which may be linear, branched, cyclic, polycyclic, unsaturated, hydroxylated, carbonylated, phosphorylated and/or sulfurized, the group containing from 1 to 24 carbon atoms and possibly having in its backbone one or more heteroatoms O, S and/or N,
the case where X═H and Z═OH being excluded.

40. Method for encapsulating a peptide active consisting of a peptide or peptide mixture and/or an amino acid or amino acid mixture, the method comprising the following successive steps:
a step of providing a mixture comprising:
an aqueous phase in which the peptide active is dissolved, optionally by means of a water-miscible co-solvent and/or a pH adjuster,
a wax phase that is solid at room temperature, consisting essentially of one or more waxes; and
an oily phase that is liquid at room temperature, consisting essentially of one or more oils,
the mixture also comprising a system of at least one non-ionic surfactant (TA1) with an HLB substantially greater than or equal to 10 and each phase being rendered homogeneous and liquid where appropriate optionally by heating and/or by mechanical and/or physical means; and
a step of hot forming an emulsion of the mixture;
a step of reducing the particle size of the emulsion to a substantially submicron size; and
a cooling step in order to form a suspension of submicron-sized structured lipid particles in which the peptide active agent is being encapsulated.

41. The method according to claim 40, wherein a ratio of weight percentage of wax to weight percentage of oil is comprised between 1:1 and 1:10.

42. The method according to claim 40, characterized by a second surfactant (TA2) with an HLB substantially less than or equal to 6.

43. The method according to claim 40, wherein the wax is chosen from cetyl palmitate, glyceryl tribehenate, glyceryl stearate or tristearate or a wax of plant or animal origin.

44. The method according to claim 40, wherein the weight percentage of wax is from 1% to 15% relative to the total weight of the mixture or suspension.

45. The method according to claim 40, wherein the weight percentage of oil is from 2% to 20% relative to the total weight of the mixture or suspension.

46. The method according to claim 40, wherein the weight percentage of peptide active is between 0.001% and 0.5%, relative to the total weight of the mixture or suspension.

47. The method according to claim 40, comprising an additional step at the end of the method, in which a second peptide compound is added to the structured particle suspension.

48. Aqueous suspension comprising the submicron-sized structured lipid particles of claim 30.

49. The suspension according to claim 48, wherein the polydispersity index is substantially less than or equal to 2 with a mean particle size ranging from 0.1 to 0.9 μm.

50. Composition for treating the skin and its integuments, comprising as an active ingredient an effective amount of the aqueous suspension of claim 49 and a physiologically acceptable vehicle.


SEQUENCE LISTING (10 sequences)

SEQ ID NO: 1 - protein, 4 aa, synthetic construct
  REGION 1..4: note = Synthetic peptide
  MOD_RES 1: note = acylation by a Palmitoyl chain on the N terminal end
GQPR

SEQ ID NO: 2 - protein, 4 aa, synthetic construct
  REGION 1..4: note = Synthetic peptide
  MOD_RES 1: note = acylation by a Palmitoyl chain on the N terminal end
KTFK

SEQ ID NO: 3 - protein, 5 aa, synthetic construct
  REGION 1..5: note = synthetic peptide
  MOD_RES 1: note = acylation by a Palmitoyl chain on the N terminal end
KTTKS

SEQ ID NO: 4 - protein, 6 aa, synthetic construct
  REGION 1..6: note = synthetic peptide
  MOD_RES 1: note = acylation by a Palmitoyl chain on the N terminal end
VGVAPG

SEQ ID NO: 5 - protein, 5 aa, synthetic construct
  REGION 1..5: note = synthetic peptide
  MOD_RES 1: note = acylation by a Palmitoyl chain on the N terminal end
KTSKS

SEQ ID NO: 6 - protein, 4 aa, synthetic construct
  MOD_RES 1: note = Acylation by a Palmitoyl chain on the N terminal end
  REGION 1..4: note = synthetic peptide
LLAN

SEQ ID NO: 7 - protein, 5 aa, synthetic construct
  REGION 1..5: note = synthetic peptide
  MOD_RES 1: note = acylation by a Palmitoyl chain on the N terminal end
YGGFL

SEQ ID NO: 8 - protein, 5 aa, Synthetic construct
  REGION 1..5: note = Synthetic peptide
KTTKS

SEQ ID NO: 9 - protein, 5 aa, Synthetic construct
  REGION 1..5: note = Synthetic peptide
  MOD_RES 1: note = Acylation by a lauryl chain on the N-terminal end
KTSKS

SEQ ID NO: 10 - protein, 5 aa, Synthetic construct
  REGION 1..5: note = Synthetic peptide
  MOD_RES 1: note = Acylation by a pentynoic chain on the N-terminal end
KTTKS